(19) United States
(12) Patent Application Publication
(10) Pub. No.: US 2002/0179457 A1
(43) Pub. Date: Dec. 5, 2002

(54) Electrochemical method for high-throughput screening of minute quantities of candidate compounds

(76) Inventor: Adam Heller, Austin, TX (US)

Correspondence Address: REED & ASSOCIATES, 800 MENLO AVENUE, SUITE 210, MENLO PARK, CA, 94025, US

(21) Appl. No.: 09/860,661

(22) Filed: May 18, 2001

(51) Int. Cl. C12Q 1/26; C12Q 1/00

(52) U.S. Cl. 205/775; 205/777.5

(57) ABSTRACT

An electrochemical method is provided for successively assessing the efficacy of each of a plurality of candidate compounds by determining the degree to which each compound directly or indirectly affects the rate of a target molecule-catalyzed electrochemical reaction. The method involves successively introducing samples containing candidate compounds into the detection chamber of a flow cell sized to contain up to about 100 μl of liquid, measuring the rate of the target molecule-catalyzed electrochemical reaction, flushing the flow cell with a carrier after each measurement, and determining the efficacy of each candidate compound based on the measured rate of the target molecule-catalyzed electrochemical reaction. The method is useful for detecting very small quantities of potential active agents, on the order of 0.1 pg. Ideally, a nonleachable redox mediator is disposed on the working electrode so as to facilitate transfer of electrons between the target molecule and the working electrode surface. The method may be employed to assess the capability of candidate compounds as enzyme inhibitors or as ligands, e.g., as receptor-binding ligands.

### TECHNICAL FIELD

This invention relates to methods for screening compounds to assess their potential utility as biologically and/or chemically active agents. More particularly, the invention relates to an electrochemical process and flow cell device for conducting such screening methods with very small samples, containing at most about one nanogram of a candidate compound. Candidate compounds include potential pharmacologically active agents such as enzyme inhibitors and receptor-binding ligands, as well as biologically or chemically active agents useful in agricultural products.

### BACKGROUND

Combinatorial processes have made possible the synthesis of very large numbers of candidate compounds that need to be screened for their activity, e.g., as enzyme inhibitors, as receptor-binding ligands, or the like. In general, these processes may involve parallel synthesis of diverse compounds by sequential addition of reagents that leads to the generation of large chemical libraries having molecular diversity. Thus, combinatorial chemistry typically involves the systematic and repetitive, covalent connection of a set of different “building blocks” of varying structures to yield large arrays of diverse candidate compounds. Such combinatorial processes have been disclosed in a number of patents. See, e.g., U.S. Pat. Nos. 5,982,387 to Hollinshead and 5,859,190 to Meyer et al.

Since combinatorial synthesis produces very large numbers of candidate compounds in quantities that are quite small, there is a need in the art for a rapid, high-throughput screening method capable of evaluating very large numbers of compounds in minuscule sample sizes.

Previous screening methods have involved assessing enzyme inhibition using dissolved enzymes, or photonic, usually calorimetric, analysis of a probed solution when the enzyme is immobilized. The activity is determined through measuring the time dependence of the concentration of a substrate of the enzyme, meaning its depletion from the solution. Alternatively, the accumulation of the product of the enzyme-catalyzed reaction may be determined. The specific binding of a candidate ligand to a receptor is usually measured by labeling the candidate blocking agent with a fluorescent tag and observing the luminescence of the immobilized, often membrane-bound, receptor. These methods do not, however, lend themselves to a rapid, high-throughput screening technique useful with minuscule quantities of candidate compounds.

High-throughput screening assay systems in microscale fluidic devices have been proposed. U.S. Pat. No. 6,150,180 to Parce et al., for example, describes microfluidic devices that may be employed to identify inhibitors of an enzymatic reaction through fluorescence. Sample flow in such microfluidic devices may be induced by applying voltages to various reservoirs in the devices to effect electroosmosis of electrokinesis. However, this means that the usefulness of this system is limited to the analysis of certain sample fluids in devices having correspondingly functionalized channels. In addition, such functionalized channels may be fouled with repeated use, further limiting analytical capacity of this type assay.

Thus, there is a need to for an improved method for evaluation of the biological activity of candidate compounds.

### SUMMARY OF THE INVENTION

Accordingly, it is a primary object of the invention to address the aforementioned need in the art by providing a method for rapidly and efficiently assessing the biological and/or chemical activity of each of a plurality of candidate compounds in succession, wherein the quantity of each candidate compound in any given sample is extremely small, on the order of 0.1 pg to about 1 ng.

It is another object of the invention to provide such a method using a flow cell containing a detection chamber having a capacity in the range of about 0.5 to about 100 μl, and wherein the aggregate volume of the liquid flowing through the system at any given time is accordingly in the range of about 0.5 to about 100 μl.

It is a further object of the invention to provide such a method wherein the time required for sample introduction, electrochemical measurement, and flushing of the detection chamber is on the order of 30 seconds or less.

It is another object of the invention to provide such a method wherein the activity assessed is the ability of each candidate compound to affect the catalytic activity of a target molecule.

It is still another object of the invention to provide such a method wherein the target molecule is an enzyme and the candidate compounds are potential inhibitors of the enzyme.

It is a further object of the invention to provide such a method wherein the activity assessed is the ability to bind to a ligand-binding partner such as a receptor, and the candidate compounds are potential ligands.

It is a further object of the invention to provide such methods in which such assessment is accompanied with the accuracy and precision associated with electrochemical analytical techniques.

Additional objects, advantages and novel features of the invention will be set forth in part in the description which follows, and in part will become apparent to those skilled in the art upon examination of the following, or may be learned by practice of the invention.

In a first embodiment, then, an electrochemical method is provided for successively assessing the efficacy of each of a plurality of candidate compounds. The method involves use of an electrochemical flow cell having a detection chamber adapted to contain in the range of about 0.5 μl to about 100 μl of liquid, an inlet for directing a stream of liquid into the detection chamber, and an outlet for directing liquid out of the detection chamber. The detection chamber includes a working electrode and a reference electrode, and, disposed on the working electrode, a target molecule that catalyzes an electrochemical reaction of at least one reactant. For each candidate compound that is evaluated, the efficacy assessed is the capability of the compound to directly or indirectly affect the catalytic activity of the target molecule, which can be determined by the degree to which the presence of a particular candidate compound affects the rate at which the electrochemical reaction proceeds. Initially, about 0.5 μl to about 100 μl of a sample is introduced through an inlet into the detection chamber so as to contact the target molecule. The sample is a liquid medium containing about 0.1 pg to about 1 ng of a candidate compound. Following sample introduction, the rate of the electrochemical reaction is measured, either amperometrically (e.g., by measuring current generated at the working electrode) or coulometrically. After the measurement, the detection chamber is flushed with a carrier liquid, which is allowed to flow through the detection chamber for a time at least sufficient to ensure that the chamber is substantially free of the candidate compound. The aforementioned steps—sample introduction, electrochemical measurement, and carrier liquid flush—are repeated with each of the plurality of candidate compounds in succession, with the time required for each cycle not exceeding about 30 seconds. The efficacy of each candidate compound is determined from the electrochemical measurement made with respect to that compound (or from the average of a plurality of electrochemical measurements made with respect to that compound), either during each cycle or following a plurality of cycles.

In a related embodiment, the candidate compound is a candidate ligand evaluated for its ability to bind to a ligand-binding partner, e.g., a receptor. Thus, in this case, the aforementioned method is modified so as to provide an electrochemical method for successively assessing the ability of each of a plurality of candidate ligands to bind to a ligand-binding partner. The method involves use of an electrochemical flow cell as described above, i.e., a flow cell having a detection chamber adapted to contain in the range of about 0.5 μl to about 100 μl of liquid, an inlet for directing a stream of liquid into the detection chamber, and an outlet for directing liquid out of the detection chamber. The detection chamber includes a working electrode and a reference electrode, and, disposed on the working electrode, a ligand-binding partner having an initial ligand bound thereto and, attached to the initial ligand, a redox enzyme that catalyzes an electrochemical reaction of a substrate of the redox enzyme. For each candidate ligand that is evaluated, the efficacy assessed is the capability of the ligand to displace the initial ligand from the ligand-binding partner, which can be determined by the decrease in the observed rate of the electrochemical reaction. That is, a candidate ligand that does not have affinity for the ligand-binding partner will not affect the observed rate of the electrochemical reaction, while a candidate ligand that has some affinity for the ligand-binding partner will reduce the observed rate of the electrochemical reaction to some degree (proportional to the candidate ligand's affinity for the ligand-binding partner), and a candidate ligand that has a strong affinity for the ligand-binding partner will substantially reduce the observed rate of the electrochemical reaction or even eliminate the occurrence thereof. To carry out this method, a sample having a volume in the range of about 0.5 μl to about 100 μl containing about 0.1 pg to about 1 ng of a candidate ligand is introduced through an inlet into the detection chamber. Following sample introduction, the rate of the electrochemical reaction is measured, either amperometrically or coulometrically, as above. After the measurement, the detection chamber is flushed with a carrier liquid, followed by introduction of another sample into the detection chamber. The steps of sample introduction, electrochemical measurement, and carrier liquid flush are repeated with each of the plurality of candidate ligands in succession, with the time required for each cycle not exceeding about 30 seconds.

In another embodiment, an alternative electrochemical method is provided for successively assessing the ability of each of a plurality of candidate ligands to bind to a ligand-binding partner such as a receptor. This method involves a “direct” assay instead of a “competitive” assay as just described. Accordingly, this alternative method involves (a) providing a flow cell as before, comprised of a detection chamber adapted to contain 0.5 μl to about 100 μl of liquid, an inlet for directing a stream of liquid into the detection chamber, and an outlet for directing liquid out of the detection chamber, the detection chamber including a reference electrode and a working electrode with the ligand-binding partner disposed thereon; (b) introducing about 0.5 μl to about 100 μl of a sample containing about 0.1 pg to about 1 ng of a candidate ligand through the inlet into the detection chamber, wherein the candidate ligand is bound to a redox enzyme that catalyzes an electrochemical reaction of a substrate of the redox enzyme; (c) determining whether or not the electrochemical reaction is taking place; (d) flushing the detection chamber as described above; and (e) assessing the efficacy of the candidate ligand from the determination made in step (c). That is, if the candidate ligand binds to the binding partner, the electrochemical reaction will be apparent on the surface of the working electrode. A carrier liquid is introduced as before, and the process may be repeated with each of a plurality of candidate ligands in succession. Again, the cycle time is on the order of 30 seconds or less.

### BRIEF DESCRIPTION OF THE DRAWINGS

The invention is described in detail below with reference to the following drawings, wherein like reference numerals indicate corresponding elements throughout the several views.

FIG. 1 illustrates in exploded view an electrochemical flow cell for use in conjunction with the method of the present invention.

FIG. 2 illustrates in cross sectional view the detection chamber formed within the electrochemical flow cell of FIG. 1.

FIG. 3 illustrates in exploded view an alternative electrochemical flow cell for use in conjunction with the present invention.

### DETAILED DESCRIPTION OF THE INVENTION

I. Terminology and Definitions:

Before describing the present invention in detail, it is to be understood that this invention is not limited to particular candidate compounds, target molecules, redox mediators, or flow cell designs, as such may vary. It is also to be understood that the terminology used herein is for the purpose of describing particular embodiments only, and is not intended to be limiting.

It must be noted that, as used in this specification and the appended claims, the singular forms “a,” “an” and “the” include plural referents unless the context clearly dictates otherwise. Thus, for example, reference to “a target molecule” includes a combination of two or more target molecules, reference to “a redox mediator” includes combinations of two or more redox mediators, reference to “a reactant” includes two or more different reactants, and the like.

In describing and claiming the present invention, the following terminology will be used in accordance with the definitions set out below.

“Amperometry” includes steady-state amperometry, chronoamperometry, and Cottrell-type measurements.

The term “attach” as used herein refers to either covalent or noncovalent binding. Noncovalent binding will typically involve “adsorption” such as may occur through hydrogen bonding, van der Waal's forces, polar attraction or electrostatic forces (i.e., through ionic bonding), or the like.

The term “candidate” as in “candidate compound” and “candidate ligand,” refers to a compound or ligand, respectively, that may or may not have the desired efficacy or activity that is assessed using the present methods. The present method is capable of assessing the efficacy of very small quantities of a candidate compound, on the order of 0.1 pg to about 1 ng, preferably about 0.1 pg to about 100 pg, more preferably about 0.1 pg to about 10 pg, and ideally about 0.1 pg to 1 pg.

“Coulometry” is the determination of charge passed or projected to pass during complete or nearly complete electrolysis of a compound, either directly on the electrode or through one or more electron transfer agents. The charge is determined by measurement of charge passed during partial or nearly complete electrolysis of the compound or, more often, by multiple measurements during the electrolysis of a decaying current and elapsed time. The decaying current results from the decline in the concentration of the electrolyzed species caused by the electrolysis.

A “counter electrode” refers to one or more electrodes paired with the working electrode, through which passes an electrochemical current equal in magnitude and opposite in sign to the current passed through the working electrode. The term “counter electrode” is meant to include counter electrodes that also function as reference electrodes (i. e. a counter/reference electrode) unless the description provides that a “counter electrode” excludes a reference or counter/reference electrode.

The “detection chamber” is defined herein as a region of the flow cell sized to contain only that portion of the sample that is to be interrogated during an assay.

An “effective diffusion coefficient” is the diffusion coefficient characterizing transport of a substance, for example, a candidate compound, a candidate ligand, an analyte, an enzyme, or a redox mediator, in the volume between the electrodes of the electrochemical cell. In at least some instances, the cell volume may be occupied by more than one medium (e.g., the sample fluid and a polymer film). Diffusion of a substance through each medium may occur at a different rate. The effective diffusion coefficient corresponds to a diffusion rate through this multiple-media volume and is typically different than the diffusion coefficient for the substance in a cell filled solely with sample fluid.

“Electrolysis” is the electrooxidation or electroreduction of a compound either directly at an electrode or via one or more electron transfer agents (e. g., redox mediators and/or enzymes).

The term “facing electrodes” refers to a configuration of the working and counter electrodes in which the working surface of the working electrode is disposed in approximate opposition to a surface of the counter electrode. In at least some instances, the distance between the working and counter electrodes is less than the width of the working surface of the working electrode.

The “flow cell” of the invention is a device configured to allow flowthrough of very small quantities of a liquid sample, on the order of 0.5 μl to 100 μl, preferably about 0.5 μl to about 10 μl, most preferably about 0.5 μl to about 1 μl, in order to detect the presence of and/or measure the concentration of a candidate compound in liquid sample via electrochemical oxidation and reduction reactions. These reactions are transduced to an electrical signal that can be correlated to an amount or concentration of the candidate molecule.

A compound is “immobilized” on a surface when it is entrapped on or chemically bound to the surface.

An “indicator electrode” includes one or more electrodes that detect partial or complete filling of a detection chamber and/or measurement zone.

The term “ligand” refers to a molecular segment or an intact molecule, generally an intact molecule, capable of attaching to a ligand-binding partner, e.g., a receptor, through either covalent or noncovalent binding.

Conversely, a ligand “binding partner” refers to a molecular segment or intact molecule, generally an intact molecule that is capable of binding a ligand either covalently or noncovalently.

A “nondiffusible,” “nonleachable,” or “non-releasable” compound is a compound that does not substantially diffuse away from the working surface of the working electrode for the duration of the electrochemical screening assay.

“Potentiometry” and “chronopotentiometry” refer to taking a potentiometric measurement at one or more points in time.

A “redox mediator” is an electron transfer agent for carrying electrons between a compound and the working electrode, either directly or indirectly.

A “reference electrode” includes a reference electrode that also functions as a counter electrode (i. e., a counter/reference electrode) unless the description provides that a “reference electrode” excludes a counter/reference electrode.

“Sorbent material” is material that wicks, retains, and/or is wetted by a fluid sample and that typically does not substantially prevent diffusion to the electrode.

A “surface in the detection chamber” includes a surface of a working electrode, counter electrode, counter/reference electrode, reference electrode, indicator electrode, a spacer, or any other surface bounding the detection chamber.

A “working electrode” is an electrode at which candidate compound is electrooxidized or electroreduced with or without the agency of a redox mediator.

A “working surface” is that portion of a working electrode that is covered with a nonleachable redox mediator and exposed to the sample, or, if the redox mediator is diffusible, a “working surface” is that portion of the working electrode that is exposed These reactions are transduced to an electrical signal that can be correlated to an amount or concentration of the candidate molecule.

The “cycle time” is the time required to introduce the sample, to perform the measurement, and to restore the system to readiness for accepting a new sample.

II. Overview:

The invention provides an electrochemical technique for conducting high-throughput screening of a plurality of candidate compounds in rapid succession, with a cycle time on the order of 30 seconds or less. The candidate compounds may be, for example, enzyme inhibitors, ligands (including receptor-binding ligands), or other compounds capable of directly or indirectly reacting with a target molecule wherein reaction with the target molecule results in an electrochemically detectable event, e.g., an increase or decrease in current. As described below, many of these candidate compounds are pharmacologically active agents.

The method involves use of an electrochemical flow cell capable of detecting extremely small quantities of a candidate compound in a sample. The volume of the sample introduced into the flow cell will not exceed about 100 μl, and may be less than about 10 μl or even less than about 1 μl. The amount of candidate compound contained in each sample introduced into the detection chamber is at most about 1 ng, but may be less than about 100 pg, and may be less than about 10 pg, or even less than about 1 pg. Generally, although not necessarily, the minimum volume of sample is about 0.5 μl and the minimum quantity of candidate compound in each sample, i.e., the minimum quantity that can be accurately and consistently detected using the present method, is about 0.1 pg. Depending upon the molecular weight of the candidate compound, quantities that are at most about 10 pmoles, preferably at most about 1 pmole, and most preferably about 0.1 pmole, can be detected. The capability of the present invention to detect such small quantities of a candidate compound is in part a result of the sensing chemistry used and in part a result of the flow cell structure and dimensions. That is, the flow cell generally comprises a thin film cell containing a detection chamber structured and sized to contain a volume of a liquid medium that is less than about than about 100 μl and that may be less than about 10 μl or even less than about 1 μl (as indicated above), which volume gives rise to a thin film that is less than about 100 μm in height, optimally less than about 50 μm in height.

The method further involves providing a flowing sample stream through a detection chamber of a flow cell. The flow is stopped periodically, and the concentration of the candidate compound is determined by an electrochemical method, such as coulometry. After the measurement, flow is resumed, thereby removing the sample the participated in the measurement from the flow cell. Alternatively, sample may flow through the chamber at a very slow rate, such that all of the candidate compound is electrolyzed in transit, yielding a current dependent only upon candidate compound concentration and flow rate. The construction of flow cells that may be used to carry out the inventive method and their operation are detailed below.

Enzyme inhibitors represent one class of candidate compounds that can be screened using the present method. Inhibitors of any enzyme can be screened, including both redox enzymes, i.e., oxidoreductases, and non-redox enzymes. The enzymes may thus be in any of the six internationally recognized enzyme classes, i.e.: oxidoreductases, which catalyze the transfer of electrons, hydride ions or hydrogen atoms; transferases, which catalyze transfer of molecular groups or segments; hydrolases, which catalyze hydrolysis reactions (transfer of functional groups to water); lyases, which catalyze the reaction or formation of double bonds; isomerases, which catalyze the transfer of atoms or groups within molecules so as to convert one isomeric form to another; and ligases, which catalyze the formation of new covalent bonds, e.g., carbon-carbon bonds. By way of example, and not limitation, inhibitors of the following specific enzymes can be assessed using the present techniques (the enzymes are listed along with their corresponding substrates and reaction product):

| | EXEMPLARY | |
|---|---|---|
| ENZYME | SUBSTRATE(S) | EXEMPLARY PRODUCT(S) |
| glucose oxidase | glucose | electrons |
| choline oxidase | choline | electrons |
| acetylcholine esterase | acetylcholine | choline |
| uricase | urate | electrons |
| cholesterol oxidase | cholesterol, | electrons |
| oxalate oxidase | oxalate, | electrons |
| superoxide dismutase | OOH radicals | H₂O₂ |
| horseradish peroxidase | H₂O₂ | electron vacancies, electrons |
| | | consumed |
| xanthine oxidase | xanthine | electrons |
| alkaline phosphatase | p-aminophenyl phosphate | p-aminophenol |
| phosphodiesterase | bis-p-aminophenyl phosphate | p-ammophenol |
| lactate dehydrogenase | lactate pyruvate, NAD or | NADH, NAD, electrons or |
| | NADH | electron vacancies |
| formate dehydrogenase | formate, NAD | NADH |
| glucose-6-phosphate | glucose-6-phosphate, NADP | NADPH |
| dehydrogenase | | |
| 6-phosphogluconate | gluconate-6-phosphate, NADP | NADPH |
| dehydrogenase | | |
| pyruvate kinase | phosphonoenol pyruvate, ADP | pyruvate, ATP |

The candidate compounds may also be ligands, screened in terms of their capability to bind to ligand-binding partners including but not limited to receptors. Receptors, as is well known in the art, are cellular macromolecules to which a compound binds in order to initiate its effects. The more studied drug receptors include, for example, cellular proteins whose normal function is to act as receptors for endogenous regulatory ligands, e.g., hormones, growth factors, neurotransmitters, and autacoids. Examples of specific receptors include, by way of illustration: plasma-bound protein kinases, which are receptors for insulin, epidermal growth factor (EGF), platelet-derived growth factor (PDGF), and certain lymphokines; cell-surface guanylyl cyclase, which is a receptor for atrial natriuretic peptides (ANP), guanylin and pheromones; neurotransmitter receptors, or ion channels, which are receptors for substances such as gamma-aminobutyric acid, glutamate, aspartate and glycine; G protein-coupled receptors, which facilitate binding of GTP to specific GTP-binding proteins (or “G proteins”); and transcription factors, which are receptors for steroid hormones, thyroid hormone, vitamin D, and retinoids.

Candidate ligands may also be screened for activity to bind to ligand-binding partners other than receptors. For example, a ligand may be screened for its activity as an antigen, to bind to an antibody, or conversely, as an antibody, to bind to an antigen. Binding of a candidate ligand to a particular binding partner (or binding of a candidate binding partner to a particular ligand) is an integral part of immunoassays, protein-binding assays, nucleic acid hybridization assays, and amplification assays. In typical immunoassays, as noted above, the ligand may be either an antigen or an antibody and the corresponding binding partner is an antibody or antigen, respectively. Various alternative immunoassay, hybridization and amplification techniques and formats thereof are well known in the art.

The sample containing the candidate compound or ligand may be derived from a biological source and will be in the form of a liquid medium in which the candidate compound is dissolved, dispersed or suspended. The liquid medium may, for example, be water, blood or urine.

III. The Flow Cell:

A flow cell is required to carry out the electrochemical assessment method of the invention. Depending on the desired electrochemical assessment, flow cell construction may differ. However, all flow cells of the invention are comprised of a detection chamber having a volume capacity in the range of about 0.5 μl to 100 μl, preferably about 0.5 μl to about 10 μl, most preferably about 0.5 μl to about 1 μl. An inlet is provided for directing a stream of liquid into the detection chamber, and an outlet is provided for directing liquid out of the detection chamber. The detection chamber also includes a working electrode, a counter electrode, and an optional reference electrode. When only two electrodes are used, the counter electrode also serves as a reference electrode. The electrodes serve to carry out the electrochemical assessment method of the invention.

It should be noted that the sensing chemistry and the construction of the flow cell both influence the accuracy and precision of electrochemical assessment, as discussed below in detail. However, as a general rule, rapid transport of species is desirable for the flow cell. Thus, cells recognized for their fast mass transport, e.g., falling film cells, may be advantageously employed. In addition, the flow through the cell can be continuous or periodically stopped. When periodically stopped, the cell is referred to a “stopped flow cell.”

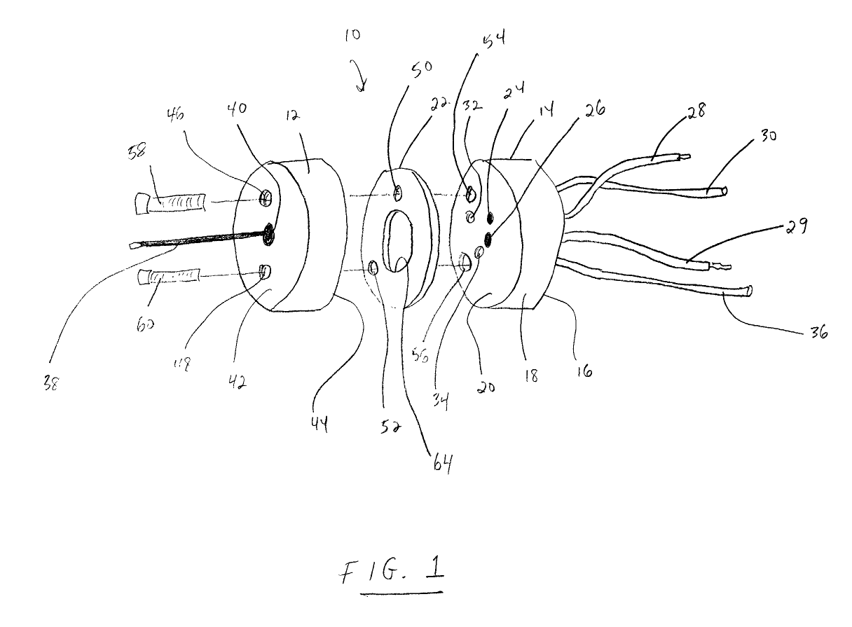

An example of an electrochemical flow cell suitable for use in carrying out the aforementioned method is illustrated in FIG. 1. As is the case with all figures referred to herein, FIG. 1 is not to scale, and, in particular, certain dimensions may be exaggerated for clarity of presentation. In the figure, the flow cell is shown generally at 10 as comprised of a working block 12 and an auxiliary block 14 each constructed of non-conducting materials such as plastics (e.g., polymethyl methacrylate, polycarbonate, polyethylene terephthalate, polystyrene, polyimide, poly(dimethylsiloxane), polypropylene or other polyolefins, etc.). As illustrated, the blocks are generally comprised of solid cylinders with planar base surfaces. For example, auxiliary block 14 is illustrated as having a generally planar base surface 16, a radially outwardly facing cylindrical side surface 18, and a generally planar axially inwardly facing mating surface 20 facing generally ring-shaped gasket 22. Working block 12 is similarly constructed.

Auxiliary block 14 includes a counter electrode 24 and a reference electrode 26, respectively coupled to a counter electrode lead 28 and a reference electrode lead 29. In an alternative embodiment, a single electrode may be present on the inner, mating surface of the auxiliary block, coupled to a single electrode lead; in such a case, the counter electrode doubles as the reference electrode. Auxiliary block 14 also houses inlet tube 30 terminating in an inlet opening 32 on mating surface 20 and, correspondingly, an outlet opening 34 in fluid communication with outlet tube 36. Working block 12 includes a working electrode 38 that is generally circular in cross-section and enters the working block at approximately the center 40 of outer surface 42 of the working block and continues through the block, terminating in a generally disk shape on the inner mating surface 44 of the working block. The exposed area of the working electrode on the mating surface 44 of the working block, which becomes one surface, generally although not necessarily the bottom surface, of the flow cell's detection chamber upon assembly of all components, is typically less than about 10⁻³ cm², preferably less than about 10⁻⁴ cm², more preferably less than about 10⁻⁵ cm².

The components of the flow cell are assembled by coupling the working block to the auxiliary block with the gasket 22 therebetween and holding the assembly together in fluid-tight alignment. One means for reversibly maintaining the assembly in fluid-tight alignment is depicted in FIG. 1, wherein axially extending apertures 46 and 48 in the working block are respectively aligned with apertures 50 and 52 in the gasket and with apertures 54 and 56 in the auxiliary block. Apertures 54 and 56 are preferably threaded so that bolts 58 and 60 can be inserted through the working block, the gasket and the auxiliary block to hold the components of the flow cell together.

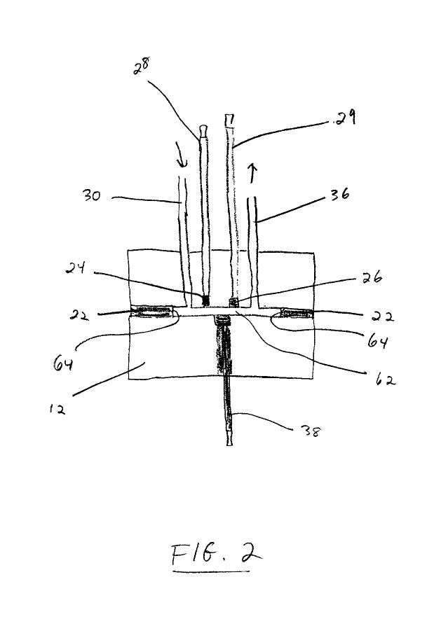

As illustrated in FIG. 2, assembly of the flow cell components in this manner results in the formation of a detection chamber 62 having a height equivalent to the thickness of the gasket 22, and bounded by the interior surface of working block 12 (including the exposed terminus of working electrode 38), the interior surface of auxiliary block 14, and the radially inwardly facing surface 64 of the gasket. As may be seen, a flow path is provided through the detection chamber in the form of a linear path, such that sample fluid introduced into the detection chamber through inlet tube 30 flows across the surface of the working electrode 38 to the outlet aperture that serves as an entrance to outlet tube 36.

It is to be understood that the aforementioned flow cell configuration and associated method of use is for the purpose of illustration, and that a variety of flow cell configurations may be employed. In addition, the flow of the liquid medium introduced into the detection chamber is not necessarily linear, but may be radial or have other types of flow paths.

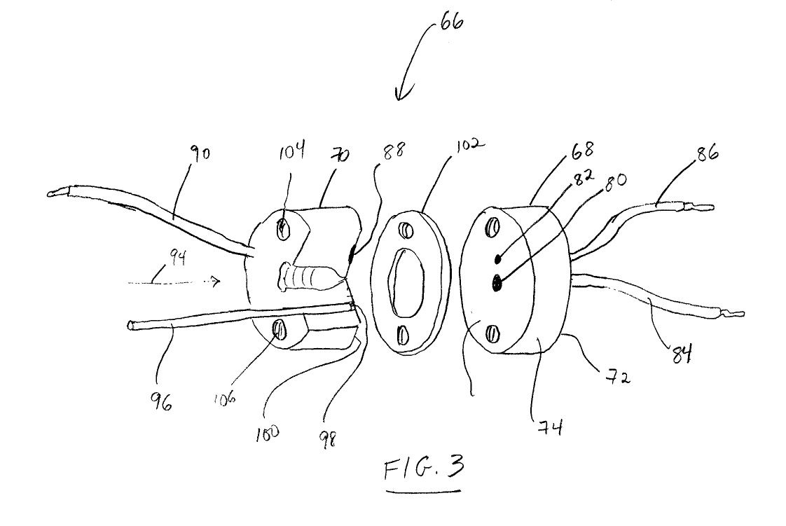

FIG. 3 illustrates an alternative flow cell that can be used in conjunction with the present method. In this figure, the flow cell is shown generally at 66 as comprised of a working block 68 and an auxiliary block 70 each constructed of non-conducting materials as described above with respect to the working and auxiliary blocks of FIG. 1. Working block 68 has a generally planar base surface 72, a radially outwardly facing cylindrical side surface 74, and a generally planar axially inwardly facing mating surface 76 facing generally ring-shaped gasket 78. As before, auxiliary block 70 is similarly constructed.

Working block 68 includes a working electrode 80 and a reference electrode 82, respectively coupled to a working electrode lead 84 and a reference electrode lead 86. In an alternative embodiment, a single electrode may be present on the inner, mating surface of the working block, coupled to a single electrode lead. Auxiliary block 70 houses counter electrode 88, coupled to counter electrode lead 90, and is provided with a central substantially cylindrical passageway 92 that serves as an inlet tube; the flow of liquid is shown at the arrow 94. The auxiliary block also houses outlet tube 96 terminating in an outlet opening 98 on mating surface 100 of the auxiliary block.

As with the flow cell illustrated in FIG. 1, the various components are assembled by coupling the working block to the auxiliary block with the gasket 102 therebetween and holding the assembly together in fluid-tight alignment. One means for reversibly maintaining the assembly in fluid-tight alignment is as depicted in FIG. 1, wherein axially extending apertures 104 and 106 in the working block are respectively aligned with apertures 108 and 110 in the gasket and with apertures 112 and 114 in the auxiliary block. Each aperture is preferably threaded so that bolts can be threaded through the auxiliary block, the gasket, and the working block as in FIG. 1.

It will be appreciated that since the fluid inlet in this embodiment terminates in the center of gasket 102, the fluid flow in the assembled flow cell will be in a direction that is generally radially outward from the center of the detection chamber formed between the auxiliary and working blocks.

IV. Flow Cell Operation:

A flow cell, either as described above or constructed according to an alternative flow cell design known in the art, is used for successively assessing: (1) the efficacy of a plurality of candidate compounds to affect the catalytic activity of a target molecule, or (2) the ability of each of a plurality of candidate ligands to bind to a ligand-binding partner. In either case, the rate or simply the occurrence of an electrochemical reaction that takes place within the detection chamber of the flow cell is used to assess the activity of a candidate compound or ligand. However, there are notable variations with respect to flow cell operation, depending on the type of candidate compound (enzyme inhibitor, ligand, etc.), the screening technique used (e.g., a direct assay versus a competitive assay), and the molecular moieties used to carry out the process (enzymes, ligand-binding partners, competitive ligands, etc.).

In assessing the capability of candidate compounds to affect the catalytic activity of a target molecule, a sample comprised of a liquid medium having a volume of less than about 100 μl, or less than about 10 μl, or even less than 1 μl, containing at most 1 ng, typically less than about 100 pg, or even less than about 10 pg or even less than about 1 pg of a candidate compound, is introduced into the detection chamber of a suitable flow cell through an inlet. A target molecule that catalyzes an electrochemical reaction of one or more reactants is disposed on the working electrode. The target molecule is preferably nonleachable but may be diffusible from the electrode in certain cases. Either during flow, or when flow is stopped, the rate of the electrochemical reaction occurring at the working electrode is measured, so that the effect of the candidate compound on the rate of reaction may be determined relative to a baseline corresponding to the rate of the reaction in the absence of a candidate compound. The effect of a candidate compound on the rate of the electrochemical reaction in turn correlates to the ability of the candidate compound to affect the catalytic activity of the target molecule. Thus, for example, a candidate compound that is an inhibitor of the target molecule's catalytic activity will decrease the rate of the electrochemical reaction. After measurement of the electrochemical reaction rate, the detection chamber is flushed by employing a suitable carrier liquid to force the sample-containing liquid medium out of the detection chamber through the outlet and into the outlet tube. Continuous flow of the carrier liquid through the detection chamber is provided for a time at least sufficient to ensure that the chamber is substantially free of the candidate compound. The aforementioned steps may be repeated with each of a number of candidate compounds in rapid succession.

Since the electrochemical reaction catalyzed by the target molecule involves the chemical transformation of one or more reactants, i.e., compounds that serve as substrates for the catalytic reaction, the operation of the flow cell in this embodiment requires such a reactant so that the electrochemical reaction catalyzed by the target molecule can occur. A sufficient amount of reactant should be maintained in the detection chamber in order to provide a baseline signal generated by the electrochemical reaction. When the method is employed to assess the efficacy of a candidate compound in enhancing the rate of the electrochemical reaction, a sufficient supply of reactant should be present in order to compensate for an enhanced rate of reaction in the presence of certain candidate compounds. In order to ensure an adequate supply of reactant through a plurality of successive cycles, the reactant is preferably introduced into the detection chamber when the sample is introduced. This often involves dissolving or suspending the reactant in the liquid medium of the sample. However, the timing and the manner of reactant introduction into the detection chamber are not critical. In some instances, the reactant may be disposed within the chamber before the introduction of the sample.

It should be noted that the reactant must be chosen according to the catalytic capability of the target molecule. Thus, for example, when the target molecule is a redox enzyme, the reactant will be a substrate of the enzyme. Upon exposure to the candidate compound, the enzyme's catalytic activity may be enhanced or suppressed. In the latter case, when a candidate compound inhibits the enzymatic activity of the target, molecule, the inhibition of the electrochemical reaction may be observed amperometrically as a drop in current or coulometrically as a drop in charge. Conversely, reaction acceleration is observed with catalytic activity enhancement. Thus, when the flow cell is used to screen the efficacy of candidate compounds to affect the catalytic activity of a redox target molecule, e.g., a redox enzyme, the rate of the electrochemical reaction of the reactant may be directly affected.

However, when the flow cell is used to screen the efficacy of candidate compounds to affect the catalytic activity of a non-redox moiety, e.g., a non-redox enzyme, the compounds' catalytic activity is assessed in an indirect manner. In this case, the reactant selected serves as a substrate for the non-redox enzyme (typically disposed on the working electrode), which catalyzes a reaction resulting in a reaction product that serves as a substrate for the redox enzyme, i.e., as a substrate for the target molecule. A cascade of enzymes may be used in this manner, with the initial enzyme transforming the reactant into a reaction product that serves as a substrate for a second enzyme, which then transforms the substrate into a reaction product that serves as a substrate for a third enzyme, and so forth. The rate of the last reaction in the cascade, caused by the target molecule's catalytic action as a redox enzyme, is dependent on the rate of the first reaction, and thus reflects the ability of a candidate compound to affect the catalytic activity of the first enzyme. Thus, turnover of the non-redox moiety is measured through a change in the concentration of its ultimate product, i.e., the final reactant produced by the cascade, which is electrolyzed (electrooxidized or electroreduced) in a reaction involving the target molecule.

More specifically, to screen the efficacy of a candidate compound to affect the catalytic activity of a non-redox moiety, a sample comprised of a liquid medium containing the candidate compound may be introduced into the detection chamber of a flow cell. The target molecule is disposed on the working electrode as well as the non-redox moiety; both the non-redox moiety and the target molecule are preferably nonleachable from the electrode. A sample comprised of a liquid medium containing a candidate compound is introduced into the detection chamber. The target molecule disposed on the working electrode serves to catalyzes an electrochemical reaction of a reactant. However, the reactant is not independently introduced or provided in the detection chamber. Instead, the reactant is produced through chemical and/or electrochemical reactions through the catalytic assistance of the non-redox moiety. Therefore, in this embodiment, one or more precursors must be provided in the detection chamber in order to assess the efficacy of each candidate compound to affect the catalytic activity of the non-redox moiety. If the candidate compound is highly effective in suppressing the catalytic activity of the non-redox moiety, then the precursor(s) will not be converted into the reactant and the electrochemical reaction involving the target molecule-catalyzed reaction of the reaction will not take place. Conversely, if the candidate is ineffective in suppressing or effective in promoting the catalytic activity of the non-redox moiety, a reactant will be formed from the precursors. In turn, the target will catalyze the electrochemical reaction of the reactant. As described above, the efficacy of the compound may be assessed either during flow, or when flow is stopped. A carrier liquid may then be used to force the sample-containing liquid medium out of the detection chamber through the outlet and into the outlet tube, and allowing continuous flow of the carrier liquid through the detection chamber for a time at least sufficient to ensure that the chamber is substantially free of the candidate compound. As a result, the aforementioned steps may be repeated with each of a number of candidate compounds in rapid succession, whether or not the candidate compounds directly or indirectly affect the catalytic activity of the target molecule.

In addition, the flow cell may be used for successively assessing the ability of each of a plurality of candidate ligands to bind to a ligand-binding partner. Such assessment can be carried out by employing either a ligand displacement technique or a ligand binding technique. In ligand displacement, a flow cell is provided as before, comprising a detection chamber that includes a working electrode, a counter electrode and an optional third electrode, and is used in conjunction with a small amount of sample containing a correspondingly small amount of candidate compound, with typical, possible and preferred amounts as described previously. Here, however, a redox enzyme, preferably a nonleachable redox enzyme, is disposed on the working electrode, wherein the redox enzyme catalyzes an electrochemical reaction of a substrate. Attached to the redox enzyme is an initial ligand having a ligand-binding partner bound thereto (e.g., a receptor having its natural ligand bound thereto). When a sample comprising a liquid medium containing a candidate ligand is introduced through the inlet into the detection chamber, the efficacy of the candidate ligand is assessed by determining the degree to which the rate of the electrochemical reaction is affected at the working electrode. That is, if the candidate ligand binds to the receptor more strongly than the initial ligand, the initial ligand will be displaced, reducing the rate of (or even entirely eliminating) the electrochemical reaction taking place at the working electrode surface. A decrease in the rate of the electrochemical reaction thus indicates the ability of a candidate ligand to bind to the ligand-binding partner, with a significant decrease in reaction rate indicating that a particular candidate ligand has substantial affinity for the receptor binding ligand. A carrier liquid is introduced to flush the detection chamber as described previously, i.e., for a time at least sufficient to ensure that the chamber is substantially free of the candidate ligand. The foregoing steps may then be repeated with each of a plurality of candidate ligands in succession in a high-throughput screening process, with at most 30 seconds between the introduction of each candidate ligand into the cell.

In contrast to the aforementioned ligand displacement technique, which is essentially a competitive assay, a ligand binding technique is a direct assay that may also be used in conjunction with the present method. In this technique, the detection chamber of the flow cell includes a working electrode with the ligand-binding partner disposed thereon. Each sample is comprised of a liquid medium containing a candidate ligand, wherein, in this embodiment, the candidate ligand is bound to a redox enzyme that catalyzes an electrochemical reaction of a substrate. The sample is introduced through the inlet into the detection chamber and the efficacy of the candidate ligand is assessed by determining whether or not the electrochemical reaction is taking place at the working electrode. That is, if the candidate ligand binds to the binding partner, the electrochemical reaction resulting from the presence of the redox enzyme on the working electrode will be apparent on the surface of the working electrode. The flow cell is flushed with a carrier liquid as before, and the process may be repeated with each of a plurality of candidate ligands in succession in a high-throughput screening process, with at most 30 seconds between the introduction of each candidate ligand into the cell.

Depending on the type of analysis and the particular analytical reagents that are used, the electrochemical reaction that is monitored occur at different electrode potentials or under different flow conditions. In some instances, the electrochemical reaction may occur upon application of approximately the same potential to the working and reference electrodes. In other cases, the electrochemical reaction may take place upon the application of a potential across the working and reference electrodes. In addition, as discussed above, electrochemical measurements may be taken either when the sample is flowing through the detection chamber or during a sample flow interruption.

V. The Working Electrode:

It should be apparent from the above description of the operation of the flow cell that the working electrode is a critical component of the flow cell for carrying out the inventive method. In general, the working electrode is small. Thus, the working surface of the electrode is also small. When circularly shaped, the electrode is typically less than about 1 mm in diameter and preferably less than 0.1 mm diameter. In addition, the working electrode may be formed from a molded carbon fiber composite. Carbon fiber composite electrodes are preferred when short measurement times are sought. Usually the diameter of the individual fibers is 1-20 μm, typically about 5-10 μm. When these are spaced such that the center-to-center distance between neighboring fibers is greater than about 10 times their diameter the capacitance is greatly reduced and the measurement times are short, typically of less than about 1 sec. The electrode may alternatively be comprised of an inert non-conducting base material, such as polyester, upon which a suitable conducting layer is deposited. The conducting layer typically has relatively low electrical resistance and should be substantially electrochemically inert over the potential range of the flow cell during operation. Suitable conducting layers include gold, carbon, platinum, ruthenium dioxide, palladium, and conductive epoxies, such as, for example, Eccocoat® CT5079-3 Carbon-Filled Conductive Epoxy Coating (available from W. R. Grace Company, Woburn, Mass.), as well as other non-corroding materials known to those skilled in the art. To form this type of electrode, the conducting layer is deposited on the surface of the inert material by any conventional method such as chemical vapor deposition or printing.

The working electrode is constructed to allow for electrical connection to external electronics such as a voltage source or current measuring equipment by providing a lead or a tip located outside the detection chamber. However, other methods or structures (such as contact pads) may also be used to connect the working electrode to the external electronics.

As described above, the working electrode must have disposed thereon a target molecule, e.g., an enzyme, and/or a ligand-binding partner. In order to monitor the electrochemical reaction that takes place at the flow cell, the target molecule and/or the ligand-binding partner must be electrically connected to the working electrode to allow electron transfer therebetween. The target molecule and/or the ligand-binding partner are typically provided in a layer that coats the working electrode. Preferably, the target molecule is nonleachable from the electrode or immobilized with respect to the polymeric layer. Covalent attachment to the polymeric layer is preferred. Optionally, the reactant involved in the reaction catalyzed by the target molecule may be included in the polymeric layer as well. This electrocatalytic coating layer may further comprise an electron conducting redox mediator that establishes an electrical connection between the electrode and the target molecule and/or the ligand-binding partner. The electron-conducting redox mediator is preferably comprised of a polymer having one or more redox species ionically, covalently or coordinatively bound thereto, in which case the redox mediator is termed a “redox polymer.” The polymer may or may not be the polymer of the aforementioned coating containing the target molecule and/or the ligand-binding partner. The inclusion of an electron conducting redox polymer is a unique feature because it may swell in the presence of the liquid medium (generally water or an aqueous solution) containing the candidate compound and thus enhance mass transport of liquid soluble matter such as ions and reactants. The liquid solubility of such matter in the redox polymer at about 25° C. should be greater than about 0.1 mM, preferably greater than about 1 mM and optimally greater than about 10 mM. Such redox polymers are described, infra.

When the present method is to screen the efficacy of candidate compounds in affecting the catalytic activity of a non-redox moiety, a plurality of compounds must be used in combination to produce a cascade of reactions, wherein the last reaction in the series is the electrochemical reaction catalyzed by the redox enzyme that serves as the target molecule. In this embodiment, the working electrode is constructed to effect and exploit such a cascade of reactions, as described in detail in part (IV). As before, the working electrode may be coated with a polymeric layer containing the target molecule. Again, covalent attachment to the polymeric layer is preferred. The non-redox moiety is preferably nonleachably disposed or immobilized on the working electrode as well, as are the various intermediate compounds, e.g., enzymes, involved in the cascade.

To prevent electrochemical reactions from occurring on portions of the working electrode not coated by the redox mediator when a nonleachable mediator is used, a dielectric material may be deposited on the electrode over, under, or surrounding the region(s) on which the redox mediator is disposed. Suitable dielectric materials include, by way of example, waxes and non-conducting organic polymers such as polyethylene. The dielectric material may also cover a portion of the redox mediator on the electrode. Generally, the surface regions containing the redox mediator will be covered in a manner that prevents exposure of the redox mediator to the sample.

VI. The Counter Electrode:

The counter electrode may be constructed in a manner similar to that described above for the working electrode. The counter electrode may also be a counter/reference electrode. Alternatively, a separate reference electrode may be provided in contact with the detection chamber. Suitable materials for the counter/reference or reference electrode include Ag/AgCl or Ag/AgBr printed on a non-conducting base material or silver chloride on a silver metal base. For example, a chlorided tip of a silver wire may be used as a reference electrode. The same materials and methods may be used to make the counter electrode as are available for constructing the working electrode, although different materials and methods may also be used. A counter electrode lead allows for convenient connection to the external electronics, such as a coulometer, potentiostat, or other measuring device. Since the counter electrode must pass an electrochemical current equal in magnitude and opposite in sign to the current passed through the working electrode, it should be designed to allow the counter electrode to accommodate the same or greater current density than the current density capability of the working electrode.

VII. The Redox Mediator:

In addition to the working electrode, sensing chemistry materials are provided for the analysis of the candidate compound. As discussed above, the sensing chemistry materials must be selected according to the type of assessment. Thus, for example, when the present method is used for successively assessing the ability of each of a plurality of candidate compound to affect the catalytic activity of a target molecule, the sensing materials include a target molecule, a reactant, and a carrier liquid. In addition, the sensing chemistry materials preferably include a redox mediator. The redox mediator may be diffusible but is preferably nonleachable (i.e., nondiffusible) because successive introduction of the carrier liquid may reduce the amount of redox mediator in the detection chamber thereby compromising the accuracy and the precision of the electrochemical measurements.

The redox mediator, whether it is diffusible or nonleachable, serves to mediate a current between the working electrode and the target molecule to enable electrochemical analysis as provided herein. That is, the mediator provides electrical contact between the target molecule and the working electrode. Although any organic or organometallic redox species can be used as a redox mediator, one type of suitable redox mediator is a transition metal compound or complex. Examples of suitable transition metal compounds or complexes include osmium, ruthenium, iron, and cobalt compounds or complexes. In addition, metallocene derivatives, such as, for example, ferrocene, can be used. Appropriate redox mediators can be identified by one of ordinary skill in the art through routine experimentation, and/or are described in the pertinent texts and literature references. See, for example, International Patent Publication No.WO 00/20626 to Feldman et al., assigned to Therasense, Inc.

The redox mediator may be a redox polymer, i.e., a polymer bound to a redox species, wherein the redox polymer is provided as a layer on the working electrode. Preferably, the redox species is nonleachable from the polymer as a result of ionic, covalent, or coordinative binding thereto. For example, a redox species composed of a transition metal complex may be nonleachably attached to a polymer in order to form a redox polymer composed of a polymeric transition metal complex. Typically, the polymers used to form redox polymers contain nitrogen-containing heterocycles, typically five- or six-membered aromatic heterocycles such as pyrrolyl, pyrrolidinyl, pyridinyl, quinolinyl, indolyl, pyrimidinyl, imidazolyl, 1,2,4-triazolyl, tetrazolyl, such as pyridinyl, imidazolyl, or derivatives thereof. Thus, suitable polymers for complexation with redox species, such as transition metal complexes as described above, include, for example, polymers and copolymers of poly (N-vinyl imidazole) (referred to as “PVI”) and poly (4-vinyl pyridine) (referred to as “PVP”), as well as polymers and copolymers of poly (acrylic acid) or polyacrylamide that have been modified by the addition of pendant nitrogen-containing heterocycles. Modification of poly(acrylic acid) may be carried out by reaction of some or all of the carboxylic acid functionalities with, for example, an aminoalkylpyridine or an aminoalkylimidazole, such as 4-ethylaminopyridine, to form amide linkages. Suitable copolymer substituents of PVI, PVP, and poly(acrylic acid) include acrylonitrile, acrylamide, acrylhydrazide, and substituted or quaternized 1-vinyl imidazole. The copolymers can be random or block copolymers. As is the case with redox mediators, appropriate redox polymers are known in the art and can be identified by one of ordinary skill in the art through routine experimentation.

The redox mediators and/or polymer layers can be bound or otherwise immobilized on the working electrode to prevent leaching of the mediator into the sample. Immobilization may be accomplished by functionalization of the electrode surface and then chemical bonding, often covalent chemical bonding, of the redox polymer to the functional groups on the electrode surface. In addition, the redox mediator can be otherwise disposed or immobilized on the working electrode by known methods, for example, via formation of multiple ion bridges with a countercharged polyelectrolyte, by covalent attachment of the redox mediator to a polymer on the working electrode, by entrapment of the redox mediator in a matrix that has a high affinity for the redox mediator, or through bioconjugation of the redox mediator to a compound bound to the working electrode. A variety of methods may be used to immobilize a redox polymer on an electrode surface. One method is adsorptive immobilization. This method is particularly useful for redox polymers with relatively high molecular weights.

The molecular weight of a redox polymer may be increased, if desired, by cross-linking. For example, the redox polymer may contain functional groups such as, for example, hydrazide, amine, alcohol, heterocyclic nitrogen, vinyl, allyl, and/or carboxylic acid groups, that serve as reactive sites and thus enable crosslinking, using a crosslinking agent or a second polymer having reactive groups that can bind to the functional groups of the first polymer. Alternatively or additionally, the functional groups may be added by a reaction, such as, for example, quaternization of amine-containing polymers to give positively charged quaternary ammonium moieties. One example of such a process is the quaternization of PVP with bromoethylamine groups.

Suitable cross-linking agents include, for example, molecules having two or more epoxide (e. g., poly (ethylene glycol) diglycidyl ether (PEGDGE)), aldehyde, aziridine, alkyl halide, and azide functional groups or combinations thereof. When a polymer has multiple acrylate functions, it can be crosslinked with a di-or polythiol; when it has multiple thiol functions it can be crosslinked with a di- or polyacrylate. Other examples of cross-linking agents include compounds that activate carboxylic acid or other acid functional groups for condensation with amines or other nitrogen compounds. These cross-linking agents include carbodiimides or compounds with active N-hydroxysuccinimide or imidate functional groups. Yet other examples of cross-linking agents are quinones (e. g., tetrachlorobenzoquinone and tetracyanoquinodimethane) and cyanuric chloride. Still other cross-linking agents may also be used. In some embodiments, an additional cross-linking agent is not required. Further discussion and examples of cross-linking and cross-linking agents are found in the following U.S. Pat. Nos.: 5,262,035; 5,262,305; 5,320,725; 5,264,104; 5,264,105; 5,356,786; and 5,593,852.

Thus, it should be apparent that one or more of the various sensing chemistry materials may be attached to one another and/or to the working electrode by employing the aforementioned techniques. Analogous methods of “attaching” molecular moieties to each other may be used in sample preparation. For example, one embodiment of the invention requires a sample containing a candidate ligand bound to a redox enzyme that catalyzes an electrochemical reaction. In such a case, the candidate ligand can be bound to the redox enzyme using coupling techniques as described above or other techniques known in the art. For covalent binding, the preferred and most versatile technique involves the reaction of a nucleophilic moiety on the ligand (e.g., an amino, hydroxyl, or sulfhydryl group) with an electrophilic moiety on the enzyme (e.g., an aldehyde, an isocyanate, etc.), or reaction of a nucleophilic moiety on the enzyme with an electrophilic moiety on the ligand.

It should be further noted that in cases wherein certain sensing chemistry materials are leachable, either a sufficient amount of the leachable material must be provided when the screening method is initiated or the material must be replenished as assessment progresses.

VII. Engineering Considerations:

The present method is preferably carried out with a thin layer electrochemical flow cell structured so as to provide desirable mass transport to the working electrode and effective utilization of the sample in the detection chamber of the cell. The cell can have two electrodes, a working electrode and a reference electrode (which also serves as the counter electrode; in this case, it may also be referred to as a “reference/counter electrode”), or it can have three electrodes, a working electrode, a counter electrode, and a separate reference electrode. The preferred location of the working electrode is in the thin layer cell. The reference/counter electrode (in a two-electrode system) or both the reference and counter electrodes (in a three-electrode system) can be upstream or downstream from the working electrode, although the preferred location of these electrodes is downstream of the working electrode, so that the chemical flux from the reference and counter electrode reactions will not reach the working electrode. The thin layer cell can be built with working and reference electrodes facing each other Such a configuration may be achieved by providing an electrically insulating spacer of a predetermined thickness between the two electrodes. These spacers are typically less than about 200 μm and preferably less than about 100 μm in thickness. The electrodes do not need, however, to directly face each other. For example, the reference electrode may be positioned adjacent to the outlet such that it is in a downstream position relative to the flow of sample through the detection chamber. Because the currents are small, less than 10⁻⁵ amperes, the voltage drops are usually not significant and the reference/counter electrode (or separate reference and counter electrodes) can be located upstream or downstream. Either the working or the reference electrodes may serve as an indicator electrode used to confirm that the cell is full. In the alternative, an additional electrode may be provided to detect partial or complete filling of a detection chamber and/or measurement zone.

It is to be understood that while the invention has been described in conjunction with the preferred specific embodiments thereof, that the foregoing description as well as the examples which follow are intended to illustrate and not limit the scope of the invention. Other aspects, advantages and modifications within the scope of the invention will be apparent to those skilled in the art to which the invention pertains.

All patents, patent applications, and publications mentioned herein are hereby incorporated by reference in their entireties.

### EXAMPLE 1

A system with a total volume of 0.1 mL comprising a flow cell is constructed. The cell comprises a detection chamber having an interior volume of 5 μl, an inlet for directing a stream of liquid into the detection chamber, and an outlet for directing liquid out of the detection chamber. The detection chamber contains a working electrode for assessing the capability of candidate compounds for inhibiting the catalytic activity of horseradish peroxidase, a carbon paste counter electrode, and downstream from the working electrode, near the outlet, a standard calomel reference electrode. The working electrode is formed by co-depositing on a conductive substrate horseradish peroxidase and a redox polymer formed of poly(4-vinyl pyridine) partially reacted with 2-bromoethylamine and partially complexed with [Os(bpy)₂Cl]^{+/2+} where bpy=2,2′-bipyridine complex of osmium (2+/3+) and poly(4-vinyl pyridine). The horseradish peroxidase and the redox polymer are crosslinked on the substrate by periodate oxidation, whereby aldehyde functions are produced in the oligosaccharides of the peroxidase, and by adding PEGDGE, polyethylene glycol diglycidyl ether.

An aqueous pH 7 phosphate buffer comprises 0.15 M NaCl and 1 mM hydrogen peroxide. Three liquid samples are prepared, each sample 0.1 mL. The first sample contains only the liquid carrier, the second sample contains the liquid carrier and 100 pg sodium cyanide, and the third carrier contains the liquid carrier and 100 pg sodium azide.

The first sample is introduced into the flow cell through the inlet, and the working electrode is poised at +0.1 V versus the potential of the standard calomel electrode. The working electrode catalyzes the electroreduction of hydrogen peroxide to water, generating a current by the reaction 2H⁺+2e+H₂O₂→2H₂O. The electric current flowing between the electrodes provides a baseline against which enzymatically inhibited current is measured.

Then, the second sample is introduced into the flow cell through the inlet, forcing the first sample out of the outlet of the flow cell. The cyanide combines with heme centers of the horseradish peroxidase and thereby inhibit its catalytic activity. As a result, the electroreduction of hydrogen peroxide to water is inhibited, and the current passing from one electrode to another is thereby decreased. The reduction in current indicates the capability of cyanide to inhibit the catalytic activity of the horseradish peroxidase.

The first sample is introduced again into the flow cell through the inlet, thereby forcing the second sample out of the outlet of the flow cell. As cyanide is flushed out of the flow cell, the current between electrodes is increased to its original magnitude. The return of the current to its original magnitude indicates the complete removal of the second sample from the detection chamber. The entire cycle time is shorter than 30 seconds.

Then, the third sample is introduced into the flow cell. Similar to the case wherein the second sample is introduced into the detection chamber, the azide in the third sample combines with heme centers of the horseradish peroxidase and thereby inhibits the catalytic activity of the peroxidase. Thus, the third sample also inhibits the electroreduction of hydrogen peroxide to water, and the current flowing from one electrode to the other electrode is decreased. The decrease in current indicates the capability of cyanide to inhibit the catalytic activity of the horseradish peroxidase. In addition, the difference in the capability of the cyanide and azide to inhibit the catalytic capability of horseradish peroxidase is evaluated by comparing the difference between the current passed between the electrodes as the second and third samples are introduced into the detection chamber.

### EXAMPLE 2

A flow cell as described in Example 1 is provided except that additionally and nonleachably attached to the working electrode are acetylcholine esterase and choline oxidase. An aqueous carrier liquid is prepared in an oxygen-containing atmosphere to comprise the buffer of Example 1 and acetylcholine, the total volume of the liquid being less than 0.1 mL. Two samples are prepared. The first sample contains only the buffered acetylcholine solution; the second sample contains the liquid carrier and 100 pg malathion, an insecticide.

The first sample is introduced into the flow cell through the inlet, and the working electrode is poised at +0.1 V versus the standard calomel electrode. The acetylcholine in the solution is hydrolyzed by the esterase on the electrode, producing choline, which, in turn, combines with dissolved oxygen to form hydrogen peroxide. The hydrogen peroxide is electroreduced to water. An electric current is produced between the electrodes as a result. The current produced provides a baseline against which current produced as a result of enzymatic inhibition is measured.

Then, the second sample is introduced into the flow cell through the inlet, forcing the first sample out of the outlet of the flow cell. The malathion inhibits the catalytic activity of the acetylcholine esterase. As a result, the production of hydrogen peroxide is inhibited, and the current passing from one electrode to another is thereby decreased. The reduction in current indicates the capability of malathion to inhibit the catalytic activity of the acetylcholine esterase.

### EXAMPLE 3

A flow cell as described in Example 1 is provided except that the horseradish peroxidase is replaced with a biological receptor. A first solution contains pH 7 phosphate buffered 0.15 M NaCl and 1 mM hydrogen peroxide and also a 100 pg of a first peptide labeled with horseradish peroxidase. When the sample is introduced into the flow cell through the inlet, the working electrodes is held at +0.1 V versus the calomel reference electrode. If the horseradish peroxide labeled peptide binds with the biological receptor the hydrogen peroxide is electroreduced and a base-line current flows.

An second solution is now prepared comprising the above buffered hydrogen peroxide solution, but containing in addition to the horseradish peroxidase labeled first peptide also 100 pg of a second un-labeled test-peptide. A decrease in the current indicates affinity of the second test peptide for the biological receptor.

An third solution is now prepared comprising the above buffered hydrogen peroxide solution, not containing in addition to the horseradish peroxidase labeled first peptide but only 100 pg of the second un-labeled test-peptide. A decrease in the current confirms the affinity of the second test peptide for the biological receptor.

We claim:

1. An electrochemical method for successively assessing the efficacy of each of a plurality of candidate compounds, comprising:
(a) providing a flow cell comprised of a detection chamber adapted to contain in the range of about 0.5 μl to about 100 μl of liquid, an inlet for directing a stream of liquid into the detection chamber, and an outlet for directing liquid out of the detection chamber, the detection chamber comprising a working electrode and a reference electrode, and, disposed on the working electrode, a target molecule that catalyzes an electrochemical reaction of a reactant, wherein the efficacy assessed is the capability of each candidate compound to directly or indirectly affect the catalytic activity of the target molecule, thereby directly or indirectly affecting the rate at which the electrochemical reaction proceeds;
(b) introducing about 0.5 μl to about 100 μl of a sample through the inlet into the detection chamber so as to contact the target molecule, wherein the sample is comprised of a liquid medium containing about 0.1 pg to about 1 ng of a candidate compound;
(c) measuring the rate of the electrochemical reaction;
(d) introducing a carrier liquid through the inlet into the detection chamber so as to force the sample-containing liquid medium out of the detection chamber through the outlet, and allowing flow of the carrier liquid through the detection chamber for a time at least sufficient to ensure that the chamber is substantially free of the candidate compound;
(e) repeating steps (b), (c) and (d) with each of the plurality of candidate compounds in succession, wherein each cycle comprised of steps (b), (c) and (d) has a cycle time not exceeding about 30 seconds; and
(f) determining the efficacy of each candidate compound from the measurements made in (c).

2. The method of claim 1, wherein the flow cell further includes a redox mediator in the detection chamber.

3. The method of claim 2, wherein the redox mediator is disposed on the working electrode.

4. The method of claim 3, wherein the redox mediator is nonleachable.

5. The method of claim 1, wherein the volume of the sample is in the range of about 0.5 to about 10 μl.

6. The method of claim 5, wherein the volume of the sample is in the range of about 0.5 to about 1 μl.

7. The method of claim 1, wherein the quantity of candidate compound in the sample is in the range of about 0.1 pg to about 100 pg.

8. The method of claim 7, wherein the quantity of candidate compound in the sample is in the range of about is in the range of about 0.1 pg to about 10 pg.

9. The method of claim 8, wherein the quantity of candidate compound in the sample is in the range of about is in the range of about 0.1 pg to about 1 pg.

10. The method of claim 1, wherein the quantity of candidate compound in the sample is at most about 10 pmoles.

11. The method of claim 10, wherein the quantity of candidate compound in the sample is at most about 1 pmole.

12. The method of claim 11, wherein the quantity of candidate compound in the sample is at most about 0.1 pmole.

13 The method of claim 1, wherein each candidate compound is a pharmacologically active agent.

14. The method of claim 1, wherein the degree to which each candidate compound affects the rate of the electrochemical reaction is assessed amperometrically, and the rate of the electrochemical reaction is determined in step (c) by measuring a current generated at the working electrode.

15. The method of claim 1, wherein the degree to which each candidate compound affects the rate of the electrochemical reaction is assessed coulometrically.

16. The method of claim 1, wherein the target molecule is a redox enzyme.

17. The method of claim 16, wherein each candidate compound is a potential inhibitor of the redox enzyme.

18. The method of claim 16, wherein a second enzyme that catalyzes a reaction is disposed on the working electrode and produces a reaction product that serves as a substrate for the redox enzyme, and each candidate compound is assessed for its efficacy in inhibiting the catalytic activity of the second enzyme.

19. The method of claim 18, wherein the second enzyme is a non-redox enzyme.

20. The method of claim 19, wherein a plurality of enzymes are disposed on the working electrode which in combination produce a cascade of enzymatic reactions ultimately producing a reaction product that serves as a substrate for the redox enzyme, wherein each candidate compound is assessed for its efficacy in inhibiting the catalytic activity of a first of the plurality of enzymes, thereby indirectly affecting the catalytic ability of the redox enzyme.

21. The method of claim 2, wherein the redox mediator provides electrical contact between the target molecule and the working electrode.

22. The method of claim 2, wherein the redox mediator comprises a polymer and a redox species ionically, covalently or coordinatively bound to the polymer.

23. The method of claim 22, wherein the redox species is ionically bound to the polymer.

24. The method of claim 22, wherein the redox species is covalently bound to the polymer.

25. The method of claim 22, wherein the redox species is coordinatively bound to the polymer.

26. The method of claim 1, wherein the electrochemical reaction takes place upon application of a potential between the working electrode and the reference electrode.

27. The method of claim 1, wherein the flow cell further includes a third electrode.

28. The method of claim 1, further comprising introducing the reactant into the detection chamber prior to or during step (b).

29. The method of claim 1, wherein the reactant is present in the sample.

30. The method of claim 1, wherein the reactant is an enzymatic substrate.

31. The method of claim 1, wherein the target molecule catalyzes an electrochemical reaction of two or more reactants.

32. The method of claim 1, wherein the efficacy of each candidate compound is assessed during flow of the sample through the detection chamber.

33. The method of claim 1, wherein after step (b), flow through the detection chamber is stopped, and not resumed until after step (c).

34. The method of claim 1, wherein the working electrode and the reference electrode are a facing electrode pair electrically insulated from each other and separated by a predetermined distance.

35. The method of claim 1, wherein the reference electrode is positioned adjacent to the outlet, such that the reference electrode is in a downstream position relative to the flow of sample through the detection chamber.

36. The method of claim 1, wherein the exposed surface area of the working electrode is less than about 10⁻³ cm².

37. The method of claim 36, wherein the exposed surface area of the working electrode is less than about 10⁻⁴ cm².

38 The method of claim 37, wherein the exposed surface area of the working electrode is less than about 10⁻⁵ cm².

39. The method of claim 1, wherein the detection chamber is adapted to contain in the range of about 0.5 to about 10 μl of liquid.

40. The method of claim 1, wherein the detection chamber is adapted to contain in the range of about 0.5 to about 1 μl of liquid.

41. An electrochemical method for successively assessing the ability of each of a plurality of candidate ligands to bind to a ligand-binding partner, comprising:
(a) providing a flow cell comprised of a detection chamber adapted to contain in the range of about 0.5 μl to about 100 μl of liquid, an inlet for directing a stream of liquid into the detection chamber, and an outlet for directing liquid out of the detection chamber, the detection chamber comprising a working electrode and a reference electrode, and, disposed on the working electrode, a ligand-binding partner having an initial ligand bound thereto and, attached to the initial ligand, a redox enzyme that catalyzes an electrochemical reaction of a substrate, wherein the ability of a candidate ligand to displace the initial ligand from the ligand-binding partner corresponds to a decrease in the rate of the rate of the electrochemical reaction;
(b) introducing about 0.5 μl to about 100 μl of a sample through the inlet into the detection chamber, wherein the sample is comprised of a liquid medium containing about 0.1 pg to about 1 ng of a candidate ligand;
(c) measuring the rate of the electrochemical reaction;
(d) introducing a carrier liquid through the inlet into the detection chamber so as to force the sample-containing liquid medium out of the detection chamber through the outlet, and allowing flow of the carrier liquid through the detection chamber for a time at least sufficient to ensure that the chamber is substantially free of the candidate ligand; and
(e) repeating steps (b), (c) and (d) with each of a plurality of candidate ligands in succession, wherein each cycle comprised of steps (b), (c) and (d) has a cycle time not exceeding about 30 seconds; and
(f) determining the ability of each candidate ligand to bind to the ligand-binding partner from the measurements made in (c).

42. An electrochemical method for successively assessing the ability of each of a plurality of candidate ligands to bind to a ligand-binding partner, comprising:
(a) providing a flow cell comprised of a detection chamber adapted to contain in the range of about 0.5 μl to about 100 μl of liquid, an inlet for directing a stream of liquid into the detection chamber, and an outlet for directing liquid out of the detection chamber, the detection chamber comprising a reference electrode and a working electrode with the ligand-binding partner disposed thereon;
(b) introducing a sample through the inlet into the detection chamber, wherein the sample is comprised of about 0.5 μl to about 100 μl of a liquid medium containing about 0.1 pg to about 1 ng of a candidate ligand, the candidate ligand bound to a redox enzyme that catalyzes an electrochemical reaction of a substrate of the redox enzyme;
(c) determining whether or not the electrochemical reaction is taking place of at the working electrode;
(d) introducing a carrier liquid into through the inlet and into the detection chamber so as to force the sample-containing liquid medium out of the detection chamber through the outlet, and allowing flow of the carrier liquid through the detection chamber for a time at least sufficient to ensure that the chamber is substantially free of the candidate ligand;
(e) repeating steps (b), (c) and (d) with each of a plurality of candidate ligands in succession, wherein each cycle comprised of steps (b), (c) and (d) has a cycle time not exceeding about 30 seconds; and
(f) determining the ability of each candidate ligand to bind to a ligand-binding partner from the measurements made in (c).

43. The method of either claim 41 or claim 42, wherein the ligand-binding partner is a receptor.

44. The method of either claim 41 or claim 42, further including a redox mediator disposed on the working electrode.

45. The method of claim 44, wherein the redox mediator is nonleachable.

46. The method of either claim 41 or claim 42, further comprising (b′) introducing a substrate of the redox enzyme into the detection chamber, prior to (b).

47. The method of claim 46, wherein (b′) is carried out simultaneously with step (b).

48. The method of claim 46, wherein step (b′) is carried out simultaneously with step (a).

49. The method of either claim 41 or claim 42, wherein the sample further comprises a substrate of the redox enzyme.

50. The method of either claim 41 or claim 42, wherein the volume of the sample is in the range of about 0.5 to about 10 μl.

51. The method of claim 50, wherein the volume of the sample is in the range of about 0.5 to about 1 μl.

52. The method of either claim 41 or claim 42, wherein the quantity of candidate ligand in the sample is in the range of about 0.1 pg to about 100 pg.

53. The method of claim 52, wherein the quantity of candidate ligand in the sample is in the range of about is in the range of about 0.1 pg to about 10 pg.

54. The method of claim 53, wherein the quantity of candidate ligand in the sample is in the range of about is in the range of about 0.1 pg to about 1 pg.

55. The method of either claim 41 or claim 42, wherein the quantity of candidate ligand in the sample is at most about 10 pmoles.

56. The method of claim 55, wherein the quantity of candidate ligand in the sample is at most about 1 pmole.

57. The method of claim 56, wherein the quantity of candidate ligand in the sample is at most about 0.1 pmole.

58. The method of either claim 41 or claim 42, wherein the rate of the electrochemical reaction is assessed amperometrically, and the rate of the electrochemical reaction is determined in step (c) by measuring a current generated at the working electrode.

59. The method of either claim 41 or claim 42, wherein the rate of the electrochemical reaction is assessed coulometrically.

60. The method of claim 44, wherein the redox mediator provides electrical contact between the target molecule and the working electrode.

61. The method of claim 44, wherein the redox mediator comprises a polymer and a redox species ionically, covalently or coordinatively bound to the polymer.

62. The method of claim 61, wherein the redox species is ionically bound to the polymer.

63. The method of claim 61, wherein the redox species is covalently bound to the polymer.

64. The method of claim 61, wherein the redox species is coordinatively bound to the polymer.

65. The method of either claim 41 or claim 42, wherein the efficacy of each candidate ligand is assessed during flow of the sample through the detection chamber.

66. The method of either claim 41 or claim 42, wherein after step (b), flow through the detection chamber is stopped, and not resumed until after step (c).

67. The method of either claim 41 or claim 42, wherein the working electrode and the reference electrode are a facing electrode pair electrically insulated from each other and separated by a predetermined distance.

68. The method of either claim 41 or claim 42, wherein the reference electrode is positioned adjacent to the outlet, such that the reference electrode is a downstream position relative to the flow of sample through the detection chamber.

69. The method of either claim 41 or claim 42, wherein the exposed surface area of the working electrode is less than about 10⁻³ cm².

70. The method of claim 69, wherein the exposed surface area of the working electrode is less than about 10⁻⁴ cm².

71. The method of claim 70, wherein the exposed surface area of the working electrode is less than about 10⁻⁵ cm².

72. The method of either claim 41 or claim 42, wherein the detection chamber further includes a sorbent material to reduce the volume of sample needed to fill the chamber.

73. The method of claim 72, wherein the sorbent material is disposed within the detection chamber in a manner that facilitates the flow of a thin film of liquid therethrough.
